(19) United States
(12) Patent Application Publication
(10) Pub. No.: US 2024/0216033 A1
(43) Pub. Date: Jul. 4, 2024

(54) BALL REMOVER

(71) Applicant: Medmetalex Co., Ltd., Osaka (JP)

(72) Inventors: Tomomi Nakagawa, Osaka (JP); Hirokazu Amino, Osaka (JP)

(21) Appl. No.: 17/920,683

(22) PCT Filed: Aug. 17, 2021
(86) PCT No.: PCT/JP2021/029987

§ 371 Date: Oct. 21, 2022

(30) Foreign Application Priority Data
Jun. 1, 2021 (JP) 2021-092314

(51) Int. Cl. A61B 17/92; A61F 2/46

(52) U.S. Cl. CPC A61B 17/92; A61F 2/4607; A61F 2002/4619

(57) ABSTRACT

A ball remover for removing a caput ball from a taper on a stem projecting from a body part of a prosthetic hip joint in the medullary cavity of the femur, comprises a handle to which an operator applies force to remove the caput ball, and a bend part extending in the medial direction at the lower end of the handle. The bend part has a forked end to accommodate the stem. A lower surface on the lateral side of the bend part remote from the median plane abuts the upper surface of a shoulder at the boundary between the body part and the stem. An upper surface of the forked end of the bend part abuts a partially flat lower surface of the caput ball and/or a surface of a lower hemisphere region of the caput ball.

### FIELD OF THE INVENTION

The present invention relates to a ball remover to remove a caput ball fitted to the neck taper of a stem of a prosthetic hip joint.

### BACKGROUND OF THE INVENTION

For a patient in whom abnormality is found in the hip joint, operations such as hip replacement arthroplasty, that replace a part of, or the entire, hip joint with a prosthetic hip joint, are conducted. Components of the prosthetic hip joint on the side of the femur comprise a stem to be inserted into the medullary cavity of the femur and a caput ball with a ball shape fitted to a tip side of a neck part of the stem.

Operations for hip replacement arthroplasty include the primary operation that installs a prosthetic hip joint into the patient's hip joint for the first time, and the revision operation that exchanges the already installed prosthetic hip joint with a new prosthetic hip joint partially or totally. In the primary operation, in order to replace the caput ball attached to the neck part once during the operation, the caput ball may need to be removed. In the revision operation, replacement of the stem is not necessary, but replacement of the components on the acetabulum side and the caput ball may be necessary. In that case, only the caput ball may need to be removed. The operation must be conducted with extreme caution in order not to damage the neck part or other parts when removing the caput ball. Especially, since a female taper part inside the caput ball and a male taper part at the tip of the neck part of the stem are very tightly taper-fitted, a very strong force or shock is required to take the caput ball off, and careful attention must be paid.

In the primary and revision operations in hip replacement arthroplasty, in order to install a prosthetic hip joint into a patient's body, the patient's body must be incised. In recent years, surgical instruments have been improved to minimize the range of the incision to reduce the burden of the patient. The invention according to Japanese Utility Model Registration No. 3165299 is devised with the aim of providing a ball remover that can prevent deterioration of workability and operability even when the incision is kept small.

### SUMMARY OF THE INVENTION

FIG. 5 of the above-referenced Utility Model Registration is reproduced herein as FIG. 13. In FIG. 13, by using an installation hole 303c formed on a femur component 303, to which an end part of a surgical instrument for installation and operation is attached, when installing the femur component 303, a support member 211 is supported by the femur component 303 without being supported by making it directly abut a surface of a neck part 303a. An energization member 212 is rotatably or pivotably supported by the support member 211, which is pivotably supported by the femur component 303. Applying impact force from a surgical instrument for hammering operation to a hammered part 225 of the energization member 212 makes an abutting part 224 in the opposite side of the hammered part 225 abut a caput ball 304 on its end surface or the like via a fulcrum to force the caput ball 304 in the direction of removal whereby the caput ball 304 is removed. The removal work of the caput ball 304 is conducted without damaging the neck part 303a of the femur component 303, and the ball remover 201 is compactly configured with the support member 211 supported with the installation hole 303c of the femur component 303, and the energization member 212 rotatably or pivotably supported by the support member 211, thereby miniaturizing the ball remover 201. Therefore, even in the case that the range of the incision is kept small, deterioration of workability and operability can be prevented.

As shown in FIG. 13, an end part of a hammering device 213b is hammered, and the force applied from the hammered part 225 is F₁. The force applied to the caput ball 304 from the abutting part 224 of the energization member 212 is F₂. When the distance from the center of a hinge pin 214 to the hammered part is b, and the perpendicular distance from the center of the hinge pin 214 in the direction of the force F₂ is a, the force F₂ is given by F₁×(b/a). Therefore, the force F₂ is smaller by (b/a) than the applied force F₁, and the applying force F₁ has to be very large to remove the caput ball 304. Applying such a stroke will not only raise a possibility of damaging the neck part 303a but also risk damaging the hinge pin 214.

The present invention addresses the above problems. The purpose of the present invention is to provide a durable ball remover that can easily remove a caput ball from a neck taper of the stem of a prosthetic hip joint in a minimally invasive operation with a small incision region.

The ball remover made to solve the above problems according to a first aspect of the present invention is a ball remover used in removing a caput ball fitted to a neck taper of a neck part projecting from the femur and arranged in a stem of a prosthetic hip joint comprising a body part inserted in the medullary cavity of the femur. The caput ball has a partially flat, notched lower end surface of ball, and a taper hole into which the neck taper is fitted. The ball remover comprises a handle part to which an operator applies force to remove the caput ball and a bend part extending to the medial direction close to the median plane of a human body that is formed at a lower end of the handle part. The bend part is characterized in that the bend part is recessed to form a fork end so as to accommodate the neck part. A lower surface of a lower end part on the lateral side of the bend part far from the median plane abuts an upper surface of a shoulder part at the boundary between the body part and the neck part. An upper surface of the fork end of the bend part abuts the lower end surface of ball of the caput ball and/or a surface at a lower hemisphere region on the side of the lower end surface of ball below the equator parallel to the lower end surface of ball of the caput ball. When the handle part is pushed down toward the lateral direction, the caput ball receives the force from the upper surface of the fork end of the bend part to be removed from the neck taper with the fulcrum that is the abutting part between the lower surface of the lower end part on the lateral side of the bend part and the upper surface of the shoulder part.

The ball remover according to the first aspect of the present invention is an application of a lever mechanism by the bell crank. A force larger than the force applied by the operator is applied to the caput ball from the upper surface of the fork end of the bend part, and thereby easily removes the caput ball. Further, a hinge pin is not used as the fulcrum as the invention described in Japanese Utility Model Registration No. 3165299. Therefore, the ball remover has a durable structure even when the force is applied to remove the caput ball.

The ball remover according to a second aspect of the present invention is the ball remover according to the first aspect wherein a screw hole to insert the stem into the medullary cavity of the femur is arranged on the shoulder part of the stem, and a projection part with a substantially hemispherical shape projects on the lower surface of the lower end part on the lateral side of the bend part so as to abut an opening of the screw hole.

In the ball remover according to the second aspect of the present invention, because the projection part with a substantially hemispherical shape projects on the lower surface of the lower end part on the lateral side of the bend part so as to abut the opening of the screw hole, force can be applied using this projection part as the fulcrum, thereby resulting in more stabilized operability.

The ball remover according to a third aspect of the present invention is the ball remover according to the second aspect of the present invention, further comprising a support plate arranged between the lower surface of the lower end part on the lateral side of the bend part and the upper surface of the shoulder part. The support plate has a plate part abutting on the lower surface of the lower end part on the lateral side of the bend part instead of the upper surface of the shoulder part, and an axis part projecting from a lower surface of the plate part to be inserted into the screw hole.

Since the ball remover according to the third aspect of the present invention comprises the support plate arranged between the lower surface of the lower end part on the lateral side of the bend part and the upper surface of the shoulder part, even with a stem having no flat part on the upper surface of the shoulder part, operability can be better stabilized by providing the support plate.

The ball remover according to a fourth aspect of the present invention is the ball remover according to any one first three aspects of the present inventions, to which an accommodation plate arranged on the upper surface of the bend part can be additionally installed, characterized in that the accommodation plate has a prescribed thickness with a through hole through which the handle part penetrates, and has substantially the same shape as the shape of the fork end of the bend part at the plan view, and when the handle part is pushed down toward the lateral direction, the caput ball receives the force from the upper surface of the fork end of the accommodation plate to be removed from the neck taper.

Since the ball remover according to the fourth aspect of the present invention further has the accommodation plate, even in the case where the length of the neck part in the longitudinal direction is long, the upper surface of the fork end of the bend part can be properly abutted on the lower end surface of the ball and/or the surface at the lower hemisphere region on the side of the lower end surface of the ball below the equator parallel to the lower end surface of the ball of the caput ball.

The ball remover according to a fifth aspect of the present invention is the ball remover according to any one of the first to fourth aspects of the present invention, characterized in that a hammered part for hammering projecting from a surface on the lateral side of the handle part in the lateral direction is formed in the handle.

In the ball remover according to fifth aspect of the present invention, since the hammered part for hammering projecting from the surface on the lateral side of the handle part in the lateral direction is formed in the handle, the caput ball can be removed with impact by knocking the hammering part with a hammer or the like. If it interferes with the insertion of the accommodation plate of the fourth aspect of the present invention, a through groove ranging with the through hole of the accommodation plate is formed such that the accommodation plate can be inserted from the handle part on the lower side of the hammered part, and the accommodation plate can be used despite the hammered part.

The ball remover according to a sixth aspect of the present invention is used in removing the caput ball fitted to the neck taper of the neck part projecting from the femur and arranged in the stem of the prosthetic hip joint comprising the body part inserted in the medullary cavity of the femur. The caput ball has the partially flat, notched, lower end surface and the taper hole into which the neck taper is fitted. The ball remover has a remover body comprising a handle part to which an operator applies force to remove the caput ball, and a stopper part formed at a lower end of the handle part and extending to the medial direction close to the median plane of a human body, and a slide body that engages with the handle part and slides in the longitudinal direction of the handle part, characterized in that the slide body is recessed to form a fork end so as to accommodate the neck part. A lower surface of a lower end on the lateral side of the stopper part far from the median plane abuts on the upper surface of the shoulder part at the boundary between the body part and the neck part. An upper surface of the fork end of the slide body is allocated by the operator so as to abut the lower end surface of ball of the caput ball and/or the surface at the lower hemisphere region on the side of the lower end surface of the ball below the equator parallel to the lower end surface of the ball of the caput ball. When the handle part is pushed down toward the lateral direction, the caput ball receives the force from the upper surface of the fork end of the slide body to be removed from the neck taper with the fulcrum that is the abutting part between the lower surface of the lower end part on the lateral side of the stopper part and the upper surface of the shoulder part.

The ball remover according to the sixth aspect of the present invention has the remover body and the slide body, and has a structure where the lower surface of the lower end part of the stopper part of the remover body abuts on the upper surface of the shoulder part, the upper surface of the fork end of the slide body is allocated by the operator so as to abut on the lower end surface of the ball and/or the surface at the lower hemisphere region on the side of the lower end surface of ball below the equator parallel to the lower end surface of ball of the caput ball. When the handle part is pushed down toward the lateral direction, the caput ball receives the force from the upper surface of the fork end of the slide body to be removed from the neck taper with the fulcrum that is the abutting part between the lower surface of the lower end part on the lateral side of the stopper part and the upper surface of the shoulder part. With this structure, sliding the slide body makes handling possible even when the length of the neck part of the stem changes. In addition, the structure according to the second aspect of the present invention, the support plate according to the third aspect of the present invention, and the hammered part according to the fifth aspect of the present invention can be added to the sixth aspect of the present invention.

According to the present inventions, a durable ball remover can be provided that can easily remove a caput ball from a neck taper of a stem of a hip joint in a minimally invasive operation with a small incision region.

### BRIEF DESCRIPTION OF THE DRAWINGS

FIG. 1 is a schematic diagram illustrating a stem of a prosthetic hip joint, to which a caput ball is fitted. inserted into a femur;

FIG. 2 is a perspective view illustrating a ball remover according to a first embodiment of the invention;

FIG. 3 is a schematic diagram illustrating a situation the use of the ball remover according to the first embodiment;

FIG. 4 is a schematic diagram illustrating the movement of the caput ball as it is being removed from the neck taper in FIG. 3;

FIG. 5 is a schematic diagram illustrating a ball remover according to a second embodiment, in which view A is a longitudinal section around the bend part, view B is a front view, view C is a side view, view D is a rear view, and view E schematically shows a situation where the ball remover is used;

FIG. 6 consists of two views illustrating the ball remover according to the second embodiment, in which view A is a perspective view illustrating the whole remover, and view B is an enlarged perspective view of the vicinity of the bend part;

FIG. 7 is a schematic diagram illustrating a ball remover comprising a support plate according to a third embodiment of the invention, in which view A is a schematic diagram illustrating a situation where the ball remover is used, and view B is a schematic diagram of the stem to which the support plate is arranged when viewed from the rear side;

FIG. 8 is a diagram illustrating a ball remover comprising an accommodation plate according to a fourth embodiment of the invention, in which view A is a top view of the accommodation plate, view B is a cross section view taken on section plane A-A, view C is a partial top view illustrating a situation where a piece of the accommodation plate is attached, and view D is a cross section of the assembly in view C;

FIG. 9 is a schematic diagram illustrating a ball remover in which a hammered part is formed according to a fifth embodiment, and showing a caput ball being removed by striking a hammered part using a hammer;

FIG. 10 is schematic diagrams illustrating a ball remover according to a sixth embodiment of the invention, in which view A shows a slide body abutting a stopper part, view B shows the slide body slid and moved upward, and view C shows where a force is applied to the handle part to remove the caput ball from the position in which it is shown in view B;

FIG. 11 is a schematic diagram in which view A is a top view illustrating the slide body and view B is a side view of the slide body, partially in cross-section;

FIG. 12 is a set of Diagrams illustrating various engaging conditions between the handle part and the slide body; and

FIG. 13 is a diagram illustrating a situation where the caput ball is being removed using the ball remover described in Japanese Utility Model Registration No. 3165299.

### DETAILED DESCRIPTION

Embodiments of the present invention will be described hereinafter based on the drawings. The embodiments described below are essentially preferred examples, and are not intended to restrict the scope of the invention, its applicable materials or its applications.

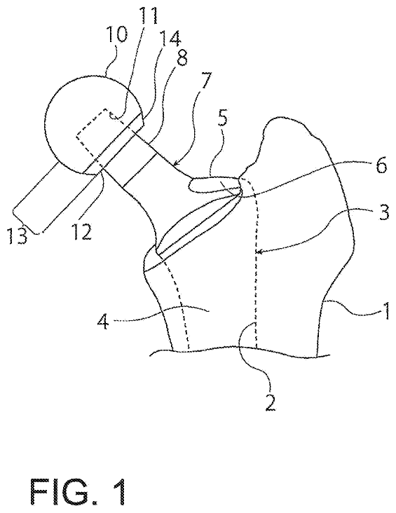

FIG. 1 is a schematic diagram illustrating a situation where a stem of a prosthetic hip joint to which a caput ball is fitted is inserted into the femur. The body part 4 of the stem 3 is inserted into the medullary cavity 2 of the femur 1, the caput ball 10, having a tapered hole 11, is fitted to the taper 8 of the neck part 7 projecting from the upper part of the femur 1. At the lower end of the caput ball 10 is a lower end surface 12. A lower hemisphere region is indicated by reference number 13. A shoulder part 6 is located between the body part 4 of the stem 3 and the neck part 7. An upper surface 5 of the shoulder part 6 is a plane.

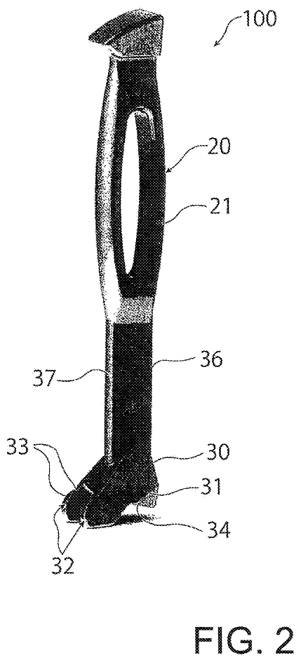

FIGS. 2 to 4 relate to the ball remover according to the first embodiment of the invention. FIG. 2 is a perspective view of the ball remover. The ball remover 100 comprises a handle part 20 having a hollow hole 21, and a bend part 30 formed at the lower end of the handle part 20.

The bend part can extend in the medial direction close to the median plane of a human body. The bend part 30 is recessed to form a forked end 32 so as to accommodate the neck part 7 of the prosthetic hip joint. The upper surface 33 of the forked end 32 is the region that is the load part that applies force to the caput ball 10. The lateral side of the lower surface 34 of the lower end part 31 of the bend part 30 is the part that serves as a fulcrum. The surface 36 on the lateral side and the surface 37 on the medial side of the handle part 20 are also shown.

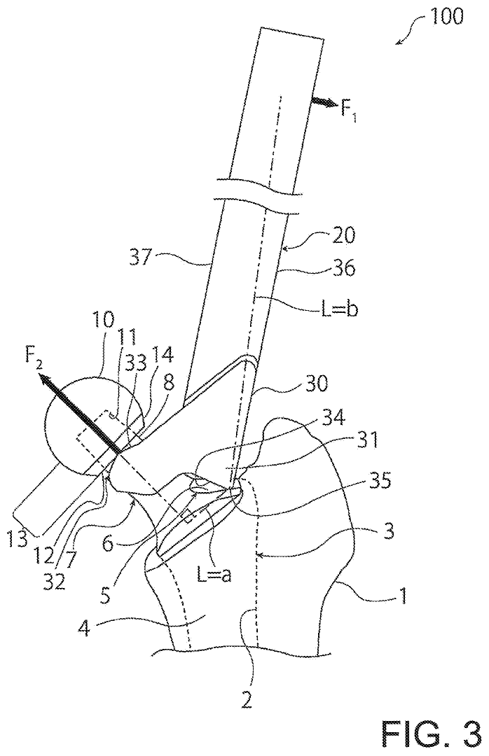

FIG. 3 shows a situation where the ball remover 100 according to the first embodiment is located in a position to start removing the ball 10. The operator grips the handle 20 to make the lower surface 34 of the lower end part abut the upper surface of the shoulder part 6, and applies a force Fa. The abutting part 35 is the fulcrum for this operation. The upper surface 33 of the fork end abuts the lower part of ball 12, and this part is the load. Further, an outer tapered surface 14 is formed between the lower end part of ball 12 and the outer spherical surface of the ball so that the load may be located on this surface.

The force F₁ applied by the operator can be compared with the force F₂ that the ball 10 receives from the ball remover 100 shown in FIG. 3, as follows. When the distance from the abutting part 35 (the fulcrum) to where the force Fᵢ is applied is b, and the distance between the abutting part 35 (the fulcrum) and the vector of the force F₂, measured in a direction perpendicular to the direction of force F₂, is a, then F₂=F₁×b/a. Therefore, the force F₂ to remove the caput ball 10 is the applying force F₁ multiplied by the coefficient b/a, and a small force can be transformed into a large load force. This principle is that of a bell crank lever mechanism, and the present invention applies this principle,

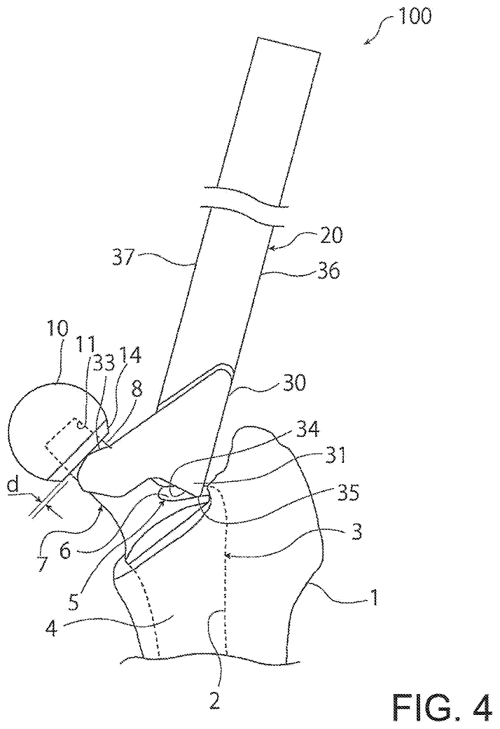

FIG. 4 is a schematic diagram illustrating the caput ball coming off from the neck taper by movement from the position shown in FIG. 3. In FIG. 4, the caput ball 10 is shifted in the axial direction of the neck taper 8 by the distance d from its position as shown in FIG. 3, resulting in a situation in which the caput ball 10 is no longer fitted to the neck taper 8.

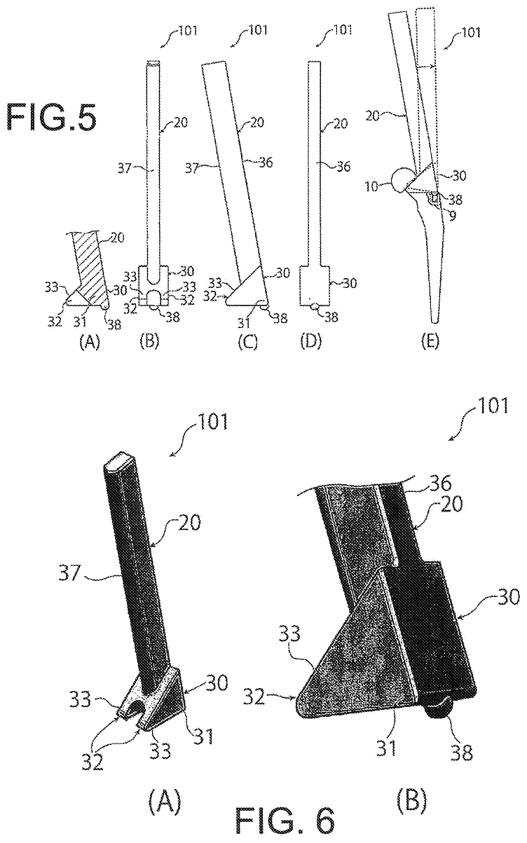

FIG. 5 shows the ball remover 101 according to the second embodiment. The same reference numerical is given to the parts that serve the same function as the first embodiment. View (A) is a longitudinal cross section view of the bend part, and this ball remover is characterized by a projection part 38 formed on the bend part. View (B) is a front view, view (C) is a side view, view (D) is a rear side view, and view (E) schematically shows the ball remover in use. In view (E), the projection part 38 enters a screw hole 9. The projection part 38 has a substantially hemisphere shape so that it moves stably according to the movement of the handle part 20.

FIG. 6 shows the ball remover according to the second embodiment. View (A) in FIG. 6 is a perspective view illustrating the whole ball remover and view (B) is an enlarged perspective view of the vicinity of the bend part.

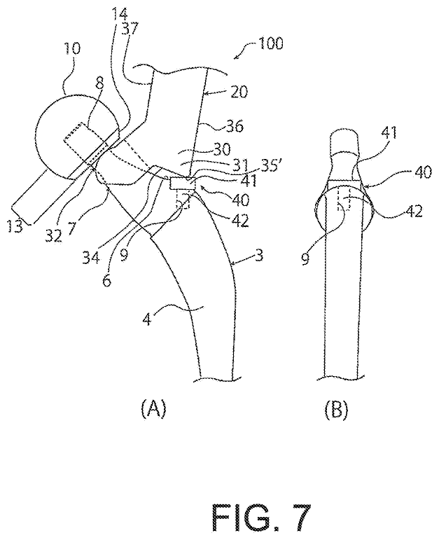

FIG. 7 shows a ball remover comprising a support plate according to a third embodiment. View (A) is a schematic diagram illustrating the ball remover in use, and view (B) is a rear schematic diagram of the stem to which the support plate is arranged. In the type of stem 3 shown in FIG. 7, the upper surface of the shoulder part 6 is not planar but curved. Therefore, the stability of the ball remover 100, in use, would be little inferior. However, to improve stability, a support plate 40 intervenes between the shoulder and the abutting part of the remover. The support plate 40 comprises a plate part 41, and an axis part 42 formed on the central part of the lower surface of the plate part. The axis part 42 is inserted into the screw hole 9. In this figure, the fulcrum is the abutting part 35′.

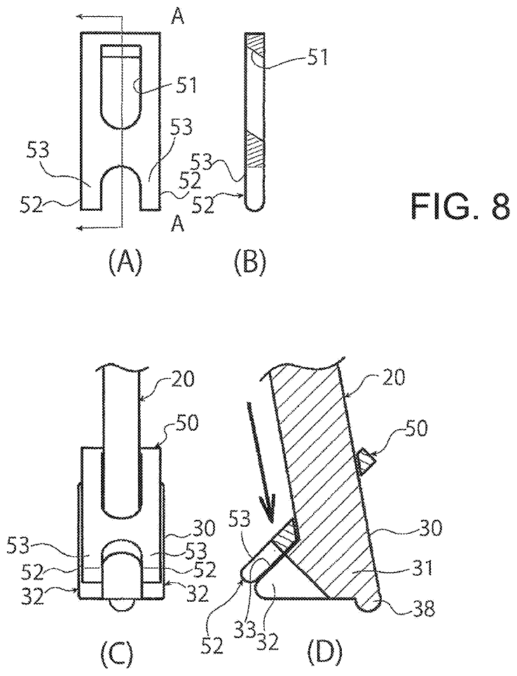

FIG. 8 shows the ball remover comprising an accommodation plate 50 according to a fourth embodiment. View (A) in FIG. 8 is a top plan view of the accommodation plate. View (B) shows a cross section view taken on section plane A-A in view A. View (C) is a partial top view illustrating the attachment of the accommodation plate 50, and view (D) is a cross section view of view (C). A through hole 51 and a forked end 52 are formed in the accommodation plate 50. The shape of the forked end 52 of the accommodation plate in view (C) has substantially the same shape as the forked end 32 of the bend part when viewed in a direction perpendicular to the upper surfaces of the accommodation plate and the bend part. The upper surface 53 of the forked end 52 of the accommodation plate abuts the caput ball 10. As shown by the arrow in view (D) of FIG. 8, the accommodation plate 50 is shifted downward along the handle part 20 and installed. Multiple plates can be installed as necessary. By using the accommodation plate 50, even in the case where the neck part is long in the longitudinal direction, the upper surface of the forked end 33 of the bend part 30 can properly abut the lower end surface of ball 12 and/or the surface at the lower hemisphere region 13 on the side of the lower end surface of ball 12 below the equator that is parallel to the lower end surface of ball 12 of the caput ball 10. In FIG. 8, although the periphery of the through hole 51 is surrounded. However, depending on the shape of the handle part 20, it is possible to form a notched groove partially in the periphery of the through hole 51, and to provide a region with a smaller cross section on the handle part 20, so that the region passes through the notched groove in the installation of the accommodation plate 50.

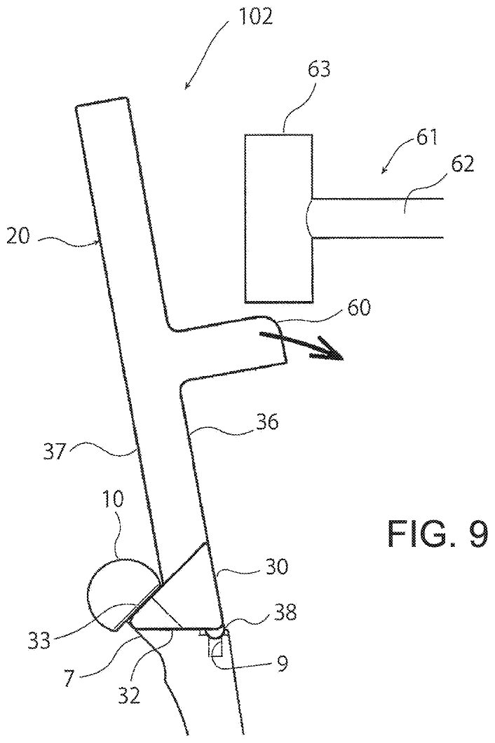

FIG. 9 shows the ball remover 102 on which a hammered part 60 is formed according to a fifth embodiment. In this embodiment, the caput ball 10 is removed by hammering the hammered part 60 using a hammer 61. The operator grips the hammer handle 62, then strikes the hammered part 60 with a hammering part 63. A significantly larger force can be instantly applied compared with the net load power by striking. Therefore, the force applied to take off the caput ball 10 can be smaller. When installing the accommodation plate 50 on this type of ball remover 102, it is necessary for a notched groove to be partially formed in the periphery of the through hole 51, and to provide a region with a smaller cross section on the handle part 20, so that the region passes through the notched groove when the accommodation plate 50 is installed.

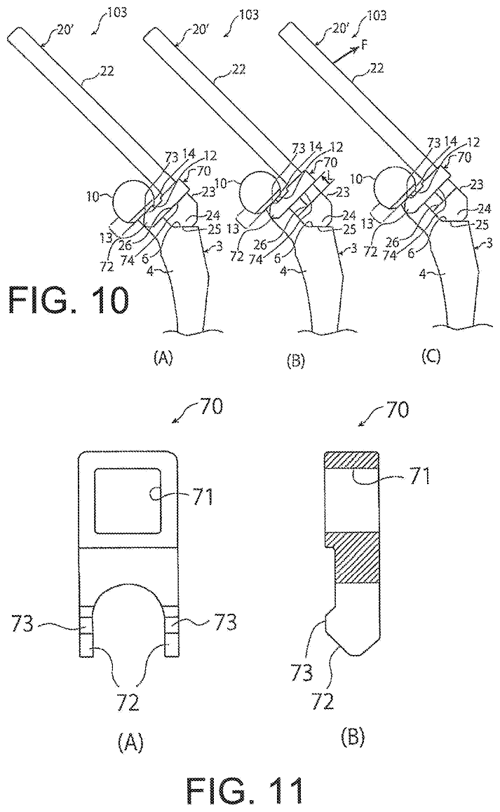

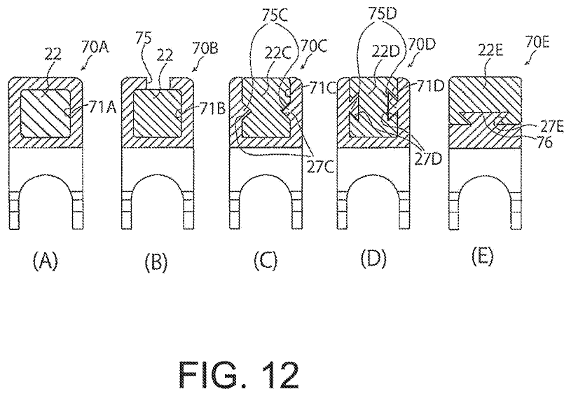

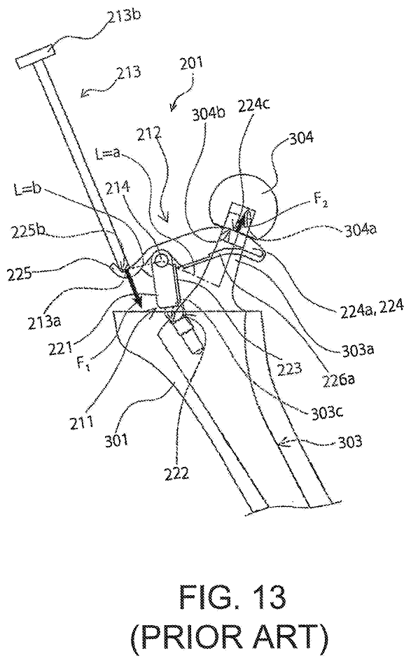

FIG. 10 shows the ball remover according to a sixth embodiment of the invention. View (A) shows a slide body 70 in abutment with a stopper part 23. This ball remover 103 has two principal parts, namely, the remover body 20′ and the slide body 70. The remover body 20′ comprises the handle part 22 and the stopper part 23. The surface on the lower side of the lower end part 24 of the stopper 23 is the lower surface of the lower end part 25, which abuts the upper surface of the shoulder 6 of the body part 4 of the stem 3. The through hole 71, shown in FIG. 11, through which the handle part 22 penetrates, is provided on the slide body 70, and the penetration of the handle part 22 through the through hole 71 allows the remover body 20′ to be engaged with the slide body 70. As an alternative to the relation between the through hole and the bar for engagement of the slide body with the handle, a groove may be formed on the handle part 22 in the longitudinal direction, and a projection extending into the groove may be formed on the slide body 70. In another alternative, a groove may be formed on the slide body 70 and a longitudinally extending projection, for extension into the groove, may be formed on the handle part 22. Specific examples of various engagement means are shown in FIG. 12. Various other means can, of course, be utilized to attain the relationship between the slide body and the handle as depicted in the several views in FIG. 12.

In view (A) of FIG. 10, since the upper surface 26 of the stopper part 26 abuts the lower surface 74 of the slide body, and the upper surface 73 of the slide body fork end abuts the lower end surface 12 of the ball, when the handle part 22 is pushed in the lateral direction, the caput ball 10 receives the force from the upper surface 73 of the fork end of the slide body 70 and is thereby removed from the neck taper (corresponding to the neck taper 8 in FIG. 1), by the action of the fulcrum at the abutment between the lower surface 25 of the lower end part 24 on the lateral side of the stopper part 23 and the upper surface of the shoulder part 6.

In view (B) of FIG. 10, compared with view (A), the neck part (corresponding to neck part 7 in FIG. 1) is longer. In this case, the operator relocates the upper surface of the forked end 73 the slide body 70 so that it engages the lower surface of ball 12 by shifting the slide body 70 upward. The arrow L indicates the distance by which the slide body is shifted by the operator.

View (C) in FIG. 10 shows a situation where the handle part 22 is pushed by applying a lateral force F, with the upper surface of the forked end of the slide body engaged with the lower surface of ball 12 by shifting of the slide body 70 upward. When the force F is applied, the caput ball 10 receives the force from the upper surface of the forked end 73 of the slide body 70 and removed from the neck taper 8 using the fulcrum formed by the abutting parts of the lower surface 25 of the lower end part 24 on the lateral side of the stopper part 23 and the upper surface of the shoulder part 6. When the force F is applied, the slide body 70 will not shift downward due to its frictional engagement with the handle part 22.

View (A) of FIG. 11 is a top view of the slide body 70 and view (B) is a side view of the slide body, partially in cross section. The slide body has a through hole 71, through which the handle part 22 extends, a forked end 72, an upper surface 73 of the forked end, and a lower surface 74.

FIG. 12 shows examples of the engagement condition between the handle part 22 and the slide body 70. The engagement conditions according to the present invention are limited to those shown in FIG. 12. Any structure in which a groove or the like is formed on either the handle part 22 or the slide body 70, and the other part has an element that falls into the groove or the like and slides therein, is sufficient.

In view (A) of FIG. 12, the transverse section of the handle part 22 is substantially square, and the through hole 71A of the slide body 70A has a substantially square shape in transverse section. This through hole, however, may have various other shapes including rectangular, polygonal, round, elliptical, a star shape, etc.

In view (B) of FIG. 12, the transverse section of the handle part 22 is substantially square, and a slide body notch 75 is formed in the slide body 70B, opening the slide body recess 71B to the exterior of the slide body. Here again, the handle part 22 may have various shapes in transverse cross-section, including rectangular, polygonal, round, elliptical, star shape, etc., and the slide body 70B may also take various shapes in accordance with the shape of the handle part. In view (B), the slide body notch 75 is disposed in a single area on one side, but it is possible to dispose it in another position.

In view (C) of FIG. 12, triangular recesses 27C are formed on the right and left sides of the handle part 22C, recess 71C is formed in the slide body 70C, and triangular projections 75C are formed in the slide body recess 71C in transverse section. The handle part recesses 27C engage with the slide body projections 75C, and accordingly the slide body 70C slides on the handle 22. The cross-sectional shape of the handle part recesses 27C and the slide body projections 75C in transverse section are not limited to triangular cross-sections, and can take various shapes including rectangular, polygonal, round, elliptical, and star shape, etc., as long as the projections and recesses have a convex-concave relationship. Further, the number of the engaging positions of the projections and recesses does not need to be two. One is sufficient as long as the projection and recess have a convex-concave relationship.

In view (D) of FIG. 12, handle part recesses 27D, each having a groove shape, are formed on the right and left sies of the handle part 22. Slide body recess 71D is formed in the slide body 70D, and slide body projections 75D are formed in the slide body recess 71D. The handle part recesses 27D engage with the slide body projections 75D, and accordingly the slide body 70D slides on the handle 22D. The handle part recesses 27D and the slide body projections 75D have a dovetail shape in transverse cross-section. However, their shape is not limited to the dovetail shape, and they can take various shapes. Further, the number of the positions at which the projections and grooves are engaged does not need to be two. One projection and one groove is sufficient.

In view (E) of FIG. 12, the handle part recess 27E with a groove shape is formed in the handle part 22E on the medial side, and a slide body projection 76 is formed in the slide body 70E in transverse section. The handle part recess 27E engages the slide body projection 76. Accordingly, the slide body 70E slides on the handle part 22E. The handle part recess 27E and the slide body projection 76 have a dovetail shape in transverse cross-section. However, the shape of the projection and groove are not limited to the dovetail shape. They can take various alternative shapes. Further, multiple cooperating projections and grooves can be utilized.

The present international application claims priority based on Japanese Patent Application 2021-92314 filed on Jun. 1, 2021, and the entire contents of Japanese Patent Application 2021-92314 are hereby incorporated into the present international application.

### INDUSTRIAL APPLICABILITY

As described above, the ball remover according to the present invention can easily remove the caput ball from the neck taper of the stem of the hip joint in a minimally invasive operation with a small incision region and is durable, and is therefore preferable for hip joint operations.

### DESCRIPTION OF THE REFERENCE NUMERALS

1 Femur
2 Medullary cavity
3 Stem
4 Body part
5 Shoulder part
6 Upper surface of shoulder part
7 Neck part
8 Neck taper
9 Screw hole
10 Caput ball
11 Taper hole
12 Lower end surface of ball
13 Lower hemisphere region
14 Outer taper surface
20, 22 Handle part
20′ Remover body
21 Hollow hole
22, 22C, 22D, 22E Handle part
23 Stopper part
24 Lower end part
25 Lower surface of lower end part
26 Upper surface of stopper part
27C, 27D, 27E Handle part recess
30 Bend part
31 Lower end part
32 Fork end
33 Upper surface of fork end
34 Lower surface of lower end part
35, 35′ Abutting part (fulcrum)
36 Surface on lateral side
37 Surface on medial side
38 Projection part
40 Support plate
41 Plate part
42 Axis part
50 Accommodation plate
51 Through hole
52 Accommodation plate fork end
53 Upper surface of accommodation plate fork end
60 Hammered part
61 Hammer
62 Hammer handle
63 Hammering part
70, 70A, 70B, 70C, 70D, 70E Slide body
71 Through hole
71A Slide body through hole
71B, 71C, 71D Slide body recess
72 Slide body fork end
73 Upper surface of slide body fork end
74 Lower surface of slide body
75 Slide body notch
75C Slide body recess projection
76 Slide body projection
100, 101, 102, 103 Ball remover

1. A ball remover used in removing a caput ball fitted to a neck taper of a neck part projecting from the femur and arranged in a stem of a prosthetic hip joint comprising a body part inserted in the medullary cavity of the femur, the caput ball having a partially flat, notched lower end surface of ball, and a taper hole into which the neck taper is fitted, the ball remover comprising:
a handle part to which an operator applies force to remove the caput ball; and
a bend part extending to the medial direction close to the median plane of a human body that is formed at a lower end of the handle part, characterized in that
the bend part is recessed to form a fork end so as to accommodate the neck part,
a lower surface of a lower end part on the lateral side of the bend part far from the median plane abuts on an upper surface of a shoulder part at the boundary between the body part and the neck part,
an upper surface of the fork end of the bend part abuts on the lower end surface of the caput ball and/or a surface at a lower hemisphere region on the side of the lower end surface of ball below the equator parallel to the lower end surface of the caput ball, and
when the handle part is pushed down toward the lateral direction, the caput ball receives the force from the upper surface of the fork end of the bend part to be removed from the neck taper with the fulcrum that is the abutting part between the lower surface of the lower end part on the lateral side of the bend part and the upper surface of the shoulder part.

2-6. (canceled)

7. The ball remover according to claim 1, wherein a screw hole to insert the stem into the medullary cavity of the femur is arranged on the shoulder part of the stem, and a projection part with a substantially hemispherical shape projects on the lower surface of the lower end part on the lateral side of the bend part so as to abut with an opening of the screw hole.

8. The ball remover according to claim 7 further comprising a support plate arranged between the lower surface of the lower end part on the lateral side of the bend part and the upper surface of the shoulder part, wherein the support plate has a plate part abutting on the lower surface of the lower end part on the lateral side of the bend part instead of the upper surface of the shoulder part and an axis part projecting from a lower surface of the plate part to be inserted into the screw hole.

9. The ball remover according to claim 1, to which an accommodation plate arranged on the upper surface of the bend part can be additionally installed, characterized in that the accommodation plate has a prescribed thickness with a through hole through which the handle part penetrates and has substantially the same shape as the shape of the fork end of the bend part at the plane view, and when the handle part is pushed down toward the lateral direction, the caput ball receives the force from the upper surface of the fork end of the accommodation plate to be removed from the neck taper.

10. The ball remover according claim 7, to which an accommodation plate arranged on the upper surface of the bend part can be additionally installed, characterized in that the accommodation plate has a prescribed thickness with a through hole through which the handle part penetrates and has substantially the same shape as the shape of the fork end of the bend part at the plane view, and when the handle part is pushed down toward the lateral direction, the caput ball receives the force from the upper surface of the fork end of the accommodation plate to be removed from the neck taper.

11. The ball remover according to claim 8, to which an accommodation plate arranged on the upper surface of the bend part can be additionally installed, characterized in that the accommodation plate has a prescribed thickness with a through hole through which the handle part penetrates and has substantially the same shape as the shape of the fork end of the bend part at the plane view, and when the handle part is pushed down toward the lateral direction, the caput ball receives the force from the upper surface of the fork end of the accommodation plate to be removed from the neck taper.

12. The ball remover according claim 1, characterized in that a hammered part for hammering projecting from a surface on the lateral side of the handle part in the lateral direction is formed in the handle.

13. The ball remover according claim 7, characterized in that a hammered part for hammering projecting from a surface on the lateral side of the handle part in the lateral direction is formed in the handle.

14. The ball remover according claim 8, characterized in that a hammered part for hammering projecting from a surface on the lateral side of the handle part in the lateral direction is formed in the handle.

15. The ball remover according claim 9, characterized in that a hammered part for hammering projecting from a surface on the lateral side of the handle part in the lateral direction is formed in the handle.

16. The ball remover according claim 10, characterized in that a hammered part for hammering projecting from a surface on the lateral side of the handle part in the lateral direction is formed in the handle.

17. The ball remover according claim 11, characterized in that a hammered part for hammering projecting from a surface on the lateral side of the handle part in the lateral direction is formed in the handle.

18. A ball remover used in removing a caput ball fitted to a neck taper of a neck part projecting from the femur and arranged in a stem of a prosthetic hip joint comprising a body part inserted in the medullary cavity of the femur, the caput ball having a partially flat, notched lower end surface of ball, and a taper hole into which the neck taper is fitted, the ball remover having:
a remover body comprising a handle part to which an operator applies force to remove the caput ball and a stopper part formed at a lower end of the handle part and extending to the medial direction close to the median plane of a human body; and
a slide body engaging with the handle part and sliding in the longitudinal direction of the handle part, characterized in that the slide body is recessed to form a fork end so as to accommodate the neck part,
a lower surface of a lower end on the lateral side of the stopper part far from the median plane abuts on an upper surface of a shoulder part at the boundary between the body part and the neck part,
an upper surface of the fork end of the slide body is allocated by the operator so as to abut on the lower end surface of the caput ball and/or the surface at the lower hemisphere region on the side of the lower end surface of ball below the equator parallel to the lower end surface of the caput ball,
and when the handle part is pushed down toward the lateral direction, the caput ball receives the force from the upper surface of the fork end of the slide body to be removed from the neck taper with the fulcrum that is the abutting part between the lower surface of the lower end part on the lateral side of the stopper part and the upper surface of the shoulder part.
